SEQUENCE LISTING (8 sequences)

SEQ ID NO: 1 - DNA, 20 bp, synthetic construct
  source 1..20: mol_type = other DNA
cccattgacgaacggaacaa

SEQ ID NO: 2 - DNA, 20 bp, synthetic construct
  source 1..20: mol_type = other DNA
tataccacgtgaattccgcc

SEQ ID NO: 3 - DNA, 20 bp, synthetic construct
  source 1..20: mol_type = other DNA
cgagaatgctaccaccttgc

SEQ ID NO: 4 - DNA, 20 bp, synthetic construct
  source 1..20: mol_type = other DNA
agccggagttcacatgtgta

SEQ ID NO: 5 - DNA, 20 bp, synthetic construct
  source 1..20: mol_type = other DNA
ctcaggtccccaatgctacc

SEQ ID NO: 6 - DNA, 20 bp, synthetic construct
  source 1..20: mol_type = other DNA
ggttgaaggccaggtagagt

SEQ ID NO: 7 - DNA, 23 bp, synthetic construct
  source 1..23: mol_type = other DNA
gcttaatttgactcaacacggga

SEQ ID NO: 8 - DNA, 24 bp, synthetic construct
  source 1..24: mol_type = other DNA
agctatcaatctgtcaatcctgtc

(19) United States
(12) Patent Application Publication
(10) Pub. No.: US 2026/0028590 A1
(43) Pub. Date: Jan. 29, 2026

(54) FERROPTOSIS INDUCING COMPOUND, COMPOSITIONS COMPRISING THE SAME, AND METHODS USING THEREOF

(71) Applicant: National Cheng Kung University, Tainan (TW)

(72) Inventors: Dar-Bin SHIEH, Tainan (TW); Pei-Wen WANG, Tainan (TW); Hsieh-Chih TSAI, New Taipei (TW); Shuian-Yin LIN, Zhubei City (TW)

(21) Appl. No.: 19/276,451

(22) Filed: Jul. 22, 2025

Related U.S. Application Data
(60) Provisional application No. 63/674,766, filed on Jul. 23, 2024.

(51) Int. Cl. C12N 5/0775; A61K 35/28

(52) U.S. Cl. CPC C12N 5/0663; A61K 35/28; C12N 5/0665; C12N 5/0667; C12N 2500/30; C12N 2500/98

(57) ABSTRACT

A method for producing an enriched population of human MSCs, comprising culturing isolated MSCs in xeno-free medium to at least 80% confluence, followed by treatment with a priming agent: erastin, sulfasalazine, or a combination thereof, to produce expanded MSCs positive for CD73, CD90, and CD105. A method is also provided for treating or preventing tissue damage or dysfunction, comprising the aforementioned method involving priming of human MSCs with a low dose of a priming agent. Further, a composition comprising primed MSCs and a cell culture medium system including the priming agent. Accordingly, the low-dose FINs offer a novel approach as a priming agent in large-scale stem cell expansion process. And the primed MSCs after low-dose FINs treatment could be applied to transplantation into oxidative and inflammatory microenvironments.

### CROSS-REFERENCE TO RELATED PATENT APPLICATION

This non-provisional application claims priority to and the benefit of, pursuant to 35 U.S.C. § 119 (a), patent application Ser. No. 63/674,766 filed in United States of America on Jul. 23, 2024. The disclosure of the above application is incorporated herein in its entirety by reference.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference were individually incorporated by reference.

### SEQUENCE LISTING

The Sequence Listing is provided as a file entitled PI-113-061-US-Sequence Listing.xml, created on Jul. 14, 2025, which is 8 kb in size. The information in the electronic format of Sequence Listing is incorporated herein by reference in its entirety.

### FIELD

The present disclosure relates to a ferroptosis inducing compound, and particularly to a ferroptosis inducer, compositions comprising the same, and methods of regulating the growth and function of mesenchymal stem cells.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Cell therapy is gaining prominence in clinical therapeutics, particularly in cancer immunotherapy, inflammatory diseases, and regenerative medicine. Mesenchymal stem cells (MSCs) are a key component of stem cell-based regenerative medicine, offering a promising approach for tissue reconstruction while addressing ethical concerns, histocompatibility issues, and the risk of teratoma formation. The versatility of adult MSCs positions them as a favored cell source in contemporary regenerative medicine.

One of the major limitations of MSC-based therapy lies in the poor survival rate of transplanted cells, primarily due to the donor or acceptor, ROS-rich microenvironment at injury sites. Additionally, the in vitro expansion of MSCs without compromising their regenerative potential remains a significant challenge in the large-scale manufacturing of cell-based therapeutics.

Accordingly, there is a need to development a compound and method using thereof to maintain stemness and efficiently proliferating MSCs to meet clinical requirements.

### SUMMARY

The present invention is made based on the discovery that a method of producing an enriched population of isolated and expanded human mesenchymal stem cells (MSCs) based on the ferroptosis inducers (FINs) precondition treatment. It is further discovered that expanded human MSCs have advantages in treating or preventing tissue damage or dysfunction associated with oxidative stress, inflammation or degenerative microenvironment condition.

An objective of the present invention is to provide a method of producing an enriched population of isolated and expanded human MSCs, the method comprising:
providing an isolated population of human MSCs;
culturing the isolated population of human MSCs in xeno-free medium, and performing subculture upon reaching at least 80% confluence to gain collected population of human MSCs; and
treating the collected population of human MSCs in the presence of priming agent to produce an enriched population of isolated and expanded human MSCs;
wherein the expanded human MSCs are positive for CD73, CD90 and CD105; and
wherein the priming agent includes erastin, sulfasalazine or a combination thereof.

In some embodiments, the isolated population of human MSCs is adult human MSCs.

In some embodiments, the isolated population of human MSCs is obtained from bone marrow, fat tissue or peripheral blood.

In some embodiments, the priming agent is configured to prime the collected population of human MSCs, to produce the primed MSCs.

In some embodiments, the erastin has a concentration of 0.1 μM to 5 μM.

In some embodiments, the sulfasalazine has a concentration of 0.0156 mM to 0.25 mM.

In some embodiments, the treating is carried out for at least 24 hours.

In some embodiments, a proliferation rate of the expanded MSCs is expanded by at least 1.4 fold after 72 hours of the treating.

In some embodiments, at least about 89.4% of the expanded MSCs are positive for CD73.

In some embodiments, at least about 92.9% of the expanded MSCs are positive for CD90.

In some embodiments, at least about 88.0% of the expanded MSCs are positive for CD105.

Another objective of the present invention also provides a method for treating or preventing tissue damage or dysfunction, comprising: administering an effective amount of primed MSCs to a target site of a subject in need thereof, wherein the primed MSCs are produced from steps, the steps comprising:
providing an isolated population of human MSCs;
culturing the isolated population of human MSCs in xeno-free medium, and performing subculture upon reaching at least 80% confluence to gain collected population of human MSCs; and
treating the collected population of human MSCs in the presence of priming agent to produce an enriched population of isolated and expanded human MSCs;
wherein the expanded human MSCs are positive for CD73, CD90 and CD105;
wherein the priming agent includes erastin, sulfasalazine or a combination thereof; and
wherein the target site of the subject is characterized by oxidative stress, inflammation or degenerative condition.

In some embodiments, the primed MSCs are administrated to the subject by a route of administration selected from the group consisting of transplantation, local injection and systemic infusion.

In some embodiments, the target site is selected from the group consisting of an osteoblast-associated site, a chondrocyte-associated site and an adipocyte-associated site.

In some embodiments, the osteoblast-associated site includes: cortical bone, trabecular bone, bone surface, periosteum, bone marrow cavity, osteogenic band or fracture healing site; wherein the chondrocyte-associated site includes: hyaline cartilage, articular cartilage, epiphyseal plate, fibrocartilage, elastic cartilage, cartilage repair site or cartilage of the respiratory tract; and wherein the adipocyte-associated site includes: white adipose tissue, subcutaneous fat, visceral fat, brown adipose tissue, bone marrow fat, fat around organs, mammary gland fat, epicardial fat or perinephric fat.

In some embodiments, the erastin has an equivalent concentration based on a range of 0.1 μM to 5 μM; and/or wherein the sulfasalazine has an equivalent concentration based on a range of 0.0156 mM to 0.25 mM.

Another objective of the present invention also provides a composition comprising primed MSCs and a cell culture medium system, wherein:
the primed MSCs are positive for CD73, CD90 and CD105; and
the cell culture medium system comprises: a xeno-free medium and a priming agent;
wherein the priming agent includes erastin, sulfasalazine or a combination thereof; and
wherein the primed MSCs are obtained from a collected population of human MSCs in the presence of the priming agent to produce primed MSCs.

In some embodiments, contacting the collected population of human MSCs with the priming agent enhances a proliferation rate and an oxidative stress tolerance when compared to without the priming agent treatment.

In some embodiments, the erastin has a concentration of 0.1 μM to 5 μM; and/or wherein the sulfasalazine has a concentration of 0.0156 mM to 0.25 mM.

In some embodiments, at least about 89.4% of the primed MSCs are positive for CD73; wherein at least about 92.9% of the primed MSCs are positive for CD90; and/or wherein at least about 88.0% of the primed MSCs are positive for CD105.

These and other aspects of the present disclosure will become apparent from the following description of the embodiment taken in conjunction with the following drawings, detailed description and the claims.

### BRIEF DESCRIPTION OF THE DRAWINGS

The accompanying drawings illustrate one or more embodiments of the disclosure and together with the written description, serve to explain the principles of the disclosure. Wherever possible, the same reference numbers are used throughout the drawings to refer to the same or like elements of an embodiment, and wherein:

FIG. 1A to 1C are diagrams illustrating cell viability assessed after 24 hours using the CCK-8 assay according to some embodiments of the present disclosure. Wherein, the FIG. 1A illustrates MSCs were treated with various concentrations of erastin. Wherein, the FIG. 1B illustrates MSCs were treated with various concentrations of sulfasalazine. Wherein, the FIG. 1C illustrates MSCs were treated with various concentrations of RSL3. Data are presented as mean±SEM from three independent experiments.

FIG. 1D is a diagram illustrating erastin activates the NRF2 pathway via p62-mediated sequestration of KEAP1, leading to antioxidant and cytoprotective responses that promotes MSCs proliferation.

FIG. 2 is a diagram illustrating dose-dependent effect of erastin on MSCs proliferation over 72 hours according to some embodiments of the present disclosure. Wherein, MSCs were treated with erastin at concentrations of 0.5 μM, 1 μM, or 5 μM, and proliferation was measured at multiple time points using the CCK-8 assay. Particularly, a significant increase in proliferation rate was observed with 5 μM erastin after 72 hours treatment, showing a 1.4-fold enhancement of proliferation rate compared to control. Data are presented as mean±SEM from three independent experiments.

FIG. 3 is a diagram illustrating erastin maintains stemness-associated gene in MSCs according to some embodiments of the present disclosure. Wherein, relative mRNA expression of stemness markers CD73, CD90, and CD105 was assessed by RT-qPCR in MSCs treated with 1 μM and 5 μM erastin for 72 hours. Data are presented as mean±SEM (n=3) and normalized to the control group. As shown in FIG. 3, there were no significant differences observed.

FIG. 4 is a diagram illustrating surface marker expression in MSCs according to some embodiments of the present disclosure. Wherein, flow cytometry analysis of MSC surface markers CD90, CD105, and CD73 after erastin treatment. Red peaks (bright peak region) represent marker-positive cells, and blue peaks (dark peak region) show isotype controls. Surface marker expression remained high across all conditions, thereby confirming that erastin treatment preserved of MSC stemness and identity.

### DETAILED DESCRIPTION

Implementations of a display module disclosed in the present disclosure are described through specific embodiments and accompanying drawings as follows. Those skilled in the art can understand the advantages and effects of the present disclosure based on the content disclosed in the specification. However, the following disclosures are not intended to limit the scope of protection of the disclosure. Under principles that do not deviate from the spirit of the present disclosure, those skilled in the art may implement the disclosure in other different embodiments based on various perspectives and applications.

In the accompanying drawings, to clearly show the components, the thicknesses of the layers, films, panels and areas, etc. are enlarged. In the disclosure, identical drawing references indicates identical components. It should be understood that components such as the layers, films, panels and areas, etc., are referred to as being “on” or “connected to” another component, they may be on or connected to another component directly, or an intermediate component may exist therebetween. To the contrary, when a component is referred to as being “directly on” or “directly connected to” another component, there is no intermediate component therebetween. As used herein, being “connected” may refer to physical connection or electrical connection.

It should be understood that terms such as “first”, “second”, and “third” are used to describe various elements, components, regions, layers and/or portions herein. However, these elements, components, regions, layers and/or parts should not be limited by these terms. These words are only used for distinguishing between an element, a component, a region, a layer and/or a part from another element, component, region, layer and/or portion. Therefore, a first “element”, “component”, “region”, “layer” and/or “portion” hereinafter may also be referred to as a second “element”, “component”, “region”, “layer” and/or “portion” without departing from the concept of the present disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

By “MSCs” it means the mesenchymal stem cells. Preferably, the MSCs are non-recombinant human mesenchymal stem cells.

In some embodiments, a population of MSCs is the adult human MSCs.

In some embodiments, human MSCs are obtained from bone marrow, fat tissue or peripheral blood, but not limited to herein.

By “low dose” it means the specific concentration of ferroptosis inducer (FIN) falls within a range that induces sub-lethal oxidative stress in cells without causing cytotoxic effects basically. The term “low dose” can be named as carefully titrated doses, sub-lethal dose or sub-lethal concentration alternatively. Besides, the low-dose of FIN shows a promotion of cell proliferation.

In some embodiments, low dose of FINs is configured to prime a collected population of human MSCs, to produce the primed human MSCs.

In some embodiments, cellular responses to oxidative stress often follow a biphasic dose-response model, commonly referred to as hormesis, in which low levels of oxidative or chemical stress enhance cellular function, while high levels induce damage or death. That is to say, reactive oxygen species (ROS) serve dual roles: at low concentrations, they function as signaling molecules that regulate processes such as proliferation and differentiation, whereas at higher concentrations, they contribute to oxidative damage and apoptosis.

In some embodiments, in terms of cellular preconditioning, where brief or sub-lethal stress exposures can prime cells to better tolerate subsequent harmful stimuli. Wherein, the cells better microenvironment adaptability is mediated by the upregulation of antioxidant defenses, enhancement of mitochondrial metabolism, or activation of cytoprotective transcription factors, but not limited to herein.

In some embodiments, the redox-tuned signaling pathways such as PI3K/AKT, MAPK/ERK, and mTOR are modulated by transient ROS elevations, promoting cell growth and survival under controlled stress conditions. These redox-adaptive mechanisms are particularly significant for mesenchymal stem cells (MSCs), which hold promise in regenerative medicine due to their immunomodulatory and tissue repair capabilities.

By “ferroptosis” it means a type of programmed cell death induced mainly by the accumulation of lipid peroxidation and lipid reactive oxygen species (ROS) in cells. The ferroptosis inducers (FINs) are divided into four classes, wherein class I includes erastin, sulfasalazine, CAY10773 or imidazole ketone erastin, wherein class II includes JKE-1674 (GPX4 Inhibitor), JKE-1716, ML-162, ML-210 or RSL3, wherein class III includes FIN56, and wherein class IV includes artemisinin, artesunate or FINO₂.

By “ferroptosis inducing compound” it means ferroptosis inducer.

In some embodiments, appropriately titrated doses of the FINs serve as mild oxidative stressors that promote adaptive responses in MSCs. It was found that low-dose FINs could promote growth, preserve stemness or maintain the proliferation of MSCs at the certain concentration.

In some embodiments, MSCs treated with low-dose FIN retained key stemness surface markers, indicating low-dose FINs in culture system preservation of their multipotent potential. As mentioned above, low-dose FINs could be incorporated into MSC culture systems to enhance expansion and cellular fitness and increase survival rates following transplantation into oxidative and inflammatory microenvironments.

The term “erastin” is a small molecule, triggering a unique iron-dependent form of nonapoptotic cell death that named as ferroptosis under certain concentration.

The term “sulfasalazine (also named as SSZ)” is an FDA-approved drug, indicated in the treatment of mild to moderate ulcerative colitis, as adjunctive therapy in severe ulcerative colitis, and for the prolongation of the remission period between acute attacks of ulcerative colitis. Furthermore, sulfasalazine induces ER stress and ferroptotic cell death under certain concentration.

In some embodiments, erastin had a concentration of, but not limited to, 0.1 μM, 0.5 μM, 1.0 μM, 1.5 μM, 2 μM, 2.5 μM, 3 μM, 3.5 μM, 4 μM, 4.5 μM or 5 μM, exhibiting no apparent cytotoxicity toward MSCs, effectively promoting their proliferation without inducing ferroptosis cell death. Preferably, erastin had a concentration of 0.1 μM to 5 μM.

In some embodiments, sulfasalazine had a concentration of, but not limited to, 0.01 mM, 0.0156 mM, 0.02 mM, 0.03 mM, 0.04 mM, 0.05 mM, 0.06 mM, 0.07 mM, 0.08 mM, 0.09 mM, 0.1 mM, 0.15 mM, 0.2 mM, 0.21 mM, 0.22 mM, 0.23 mM, 0.24 mM or 0.25 mM, exhibiting no apparent cytotoxicity toward MSCs, effectively promoting their proliferation without inducing ferroptosis cell death. Preferably, sulfasalazine had a concentration of 0.0156 mM to 0.25 mM.

In some embodiments, erastin at 0.1 μM to 5 μM and sulfasalazine at 0.0156 mM to 0.25 mM exhibited no apparent cytotoxicity toward MSCs, effectively promoting their proliferation without inducing ferroptosis cell death.

In some embodiments, treatment with ferroptosis inducers (FINs), for example erastin and/or sulfasalazine treatment, has long-term positive effects on MSC differentiation capacity, immunomodulatory function, and genomic stability.

In some embodiments, treatment with FINs, for example erastin and/or sulfasalazine treatment, positively influences mesenchymal stem cell (MSC) function by promoting lineage-specific differentiation, reinforcing immunomodulatory activity, and preserving genomic stability.

In some embodiments, treatment with FINs, for example erastin and/or sulfasalazine treatment, activates the NRF2 pathway and downstream antioxidant gene expression.

In some embodiments, transcriptomic and metabolomic profiling support the results that adaptive cellular responses triggered by low-dose FIN exposure.

In some embodiments, low-dose of erastin providing low levels of oxidative stress stimulated adaptive cellular responses that enhanced survival, proliferation, and differentiation function of MSCs. That is to say, sub-lethal oxidative stress induced by erastin activated antioxidant defense systems, notably the NRF2 pathway, through the disruption of the KEAP1-NRF2 complex via p62-mediated sequestration.

In some embodiments, treatment with low-dose FINs (such as erastin and/or sulfasalazine) in MSCs culture system, induced mild oxidative stress characterized by moderate intracellular ROS elevation without cytotoxicity. This sub-lethal oxidative stress activated the p62-KEAP1-NRF2 axis, evidenced by increased NRF2 nuclear translocation and upregulation of antioxidant response genes. As a result, primed MSCs with low-dose FINs exhibited enhanced oxidative stress tolerance, increased MSCs viability and growth, improved differentiation potential, and elevated immunomodulatory activity, thereby FINs can prime MSCs through redox-mediated adaptive reprogramming.

In some embodiments, low-dose FINs (such as erastin and/or sulfasalazine) in contact with MSCs primed them for oxidative stress tolerance, which was highly beneficial for transplantation into inflammatory or ischemic microenvironments characterized by elevated levels of ROS, and beneficial for MSCs adaptive to microenvironment of the transplantation site. Moreover, the primed MSCs with low-dose FINs exhibited improved engraftment, persistence, and therapeutic outcomes in vivo.

In some embodiments, treating a population of human MSCs in the presence of priming agent is carried out for at least 1 day, 2 day, 3 day, 4 day, 5 day, 6 day, 7 day or 8 day.

In some embodiments, a proliferation rate of the expanded MSCs is expanded by at least 1.4 fold after 72 hours of the priming agent treatment, for example erastin or sulfasalazine.

In some embodiments, a composition comprising primed MSCs and a cell culture medium system, wherein:
the primed MSCs are positive for CD73, CD90 and CD105; and
the cell culture medium system comprises: a xeno-free medium and a priming agent;
wherein the priming agent includes erastin, sulfasalazine or a combination thereof; and
wherein the primed MSCs are obtained from a collected population of human MSCs in the presence of the priming agent to produce primed MSCs.

In some embodiments, contacting the collected population of human MSCs with the priming agent enhanced a proliferation rate and an oxidative stress tolerance when compared to without the priming agent treatment.

In some embodiments, a method for treating or preventing tissue damage or dysfunction, comprising: administering an effective amount of primed MSCs to a target site of a subject in need thereof, wherein the primed MSCs are produced from steps, the steps comprising:
providing an isolated population of human MSCs;
culturing the isolated population of human MSCs in xeno-free medium, and performing subculture upon reaching at least 80% confluence to gain collected population of human MSCs; and
treating the collected population of human MSCs in the presence of priming agent to produce an enriched population of isolated and expanded human MSCs;
wherein the expanded human MSCs are positive for CD73, CD90 and CD105;
wherein the priming agent includes erastin, sulfasalazine or a combination thereof; and
wherein the target site of the subject is characterized by oxidative stress, inflammation or degenerative condition.

By “subject” it means the human being or the animals, but not limited to herein.

In some embodiments, the primed MSCs are administrated to the subject by a route of administration selected from the group consisting of transplantation, local injection and systemic infusion, but not limited to herein.

In some embodiments, the target site is selected from the group consisting of an osteoblast-associated site, a chondrocyte-associated site and an adipocyte-associated site, but not limited to herein.

In some embodiments, the osteoblast-associated site includes: cortical bone, trabecular bone, bone surface, periosteum, bone marrow cavity, osteogenic band or fracture healing site; wherein the chondrocyte-associated site includes: hyaline cartilage, articular cartilage, epiphyseal plate, fibrocartilage, elastic cartilage, cartilage repair site or cartilage of the respiratory tract; and wherein the adipocyte-associated site includes: white adipose tissue, subcutaneous fat, visceral fat, brown adipose tissue, bone marrow fat, fat around organs, mammary gland fat, epicardial fat or perinephric fat, but not limited to herein.

In some embodiments, erastin had an equivalent concentration based on a range of 0.1 μM to 5 μM; and/or wherein the sulfasalazine had an equivalent concentration based on a range of 0.0156 mM to 0.25 mM.

In some embodiments, the erastin-preconditioned MSCs hold therapeutic advantages for regenerative applications, particularly in clinical contexts characterized by oxidative stress or inflammation.

In some embodiments, low-dose of erastin preserved the stemness markers (CD73, CD90, CD105) expression, thereby erastin being as a priming agent in large-scale stem cell expansion process.

In some embodiments, after the priming agent treatment (for example erastin or sulfasalazine), at least about 89.4%, 90%, 91%, 92%, 93%, 94%, 95%, 96%, 97%, 98% or 99% of the expanded MSCs are positive for CD73. At least about 92.9%, 93%, 94%, 95%, 96%, 97%, 98% or 99% of the expanded MSCs are positive for CD90. At least about 88.0%, 89%, 90%, 91%, 92%, 93%, 94%, 95%, 96%, 97%, 98% or 99% of the expanded MSCs are positive for CD105.

### EXAMPLES

The detailed description and preferred embodiments of the invention will be set forth in the following content, and provided for people skilled in the art to understand the characteristics of the invention.

### Material and Methods

### MSC Culture and Cell Proliferation Assays

A method of producing an enriched population of isolated and expanded human mesenchymal stem cells (MSCs), the method comprising:
providing an isolated population of human MSCs;
culturing the isolated population of human MSCs in xeno-free medium, and performing subculture upon reaching at least 80% confluence to gain collected population of human MSCs; and
treating the collected population of human MSCs in the presence of priming agent to produce an enriched population of isolated and expanded human MSCs;
wherein the expanded human MSCs are positive for CD73, CD90 and CD105; and
wherein the priming agent includes erastin, sulfasalazine or a combination thereof.

TABLE 1
FINs used in MSC Culture system

| FINs | PubChem CID |
|---|---|
| erastin | 11214940 |
| sulfasalazine | 5339 |
| RSL3 | 1750826 |

RNA Isolation and Quantitative Real-Time PCR (qRT-PCR)

Total RNA was extracted from MSCs using Genezol™ RNA isolation reagent (Geneaid, Taiwan), following the manufacturer's instructions. Quantitative real-time PCR was performed using KAPA SYBR FAST One-Step qRT-PCR Master Mix (Kapa Biosystems, USA) on a StepOnePlus Real-Time PCR System (Applied Biosystems, USA). The relative expression levels of target genes were calculated using the 2\hat{ }-ΔΔCt method, normalized to 18S rRNA. Statistical comparisons of gene expression levels across treatment groups were performed using multiple unpaired t-tests in GraphPad Prism v9.3.1, with p-values adjusted via the Bonferroni-Dunn method. A threshold of p<0.05 was considered statistically significant.

TABLE 2
Primer sequences used in qRT-PCR

| Primer | |
|---|---|
| name | Sequence (5′→3′) |
| CD73 | Forward: CCCATTGACGAACGGAACAA |
| | (SEQ ID NO: 1) |
| | Reverse: TATACCACGTGAATTCCGCC |
| | (SEQ ID NO: 2) |
| CD90 | Forward: CGAGAATGCTACCACCTTGC |
| | (SEQ ID NO: 3) |
| | Reverse: AGCCGGAGTTCACATGTGTA |
| | (SEQ ID NO: 4) |
| CD105 | Forward: CTCAGGTCCCCAATGCTACC |
| | (SEQ ID NO: 5) |
| | Reverse: GGTTGAAGGCCAGGTAGAGT |
| | (SEQ ID NO: 6) |
| 18S rRNA | Forward: GCTTAATTTGACTC AACACGGGA |
| | (SEQ ID NO: 7) |
| | Reverse: AGCTATCAATCTGTCAATCCTGTC |
| | (SEQ ID NO: 8) |

### Flow Cytometry for MSC Surface Marker Analysis

To assess MSC surface marker expression, cells treated with different erastin concentrations were harvested and resuspended in FACS buffer at 5×10⁶ cells/mL, followed by filtration through a 70 μm strainer. Surface marker profiling was performed using the Human MSC Analysis Kit (BD Biosciences, USA), which includes antibodies against CD73 (APC), CD90 (FITC), and CD105 (PerCP-Cy5.5). Cells were incubated with antibody cocktails for 30 minutes in the dark at room temperature, washed, and resuspended in FACS buffer. Isotype-matched controls were included using PE-conjugated antibody cocktails. Each sample and control tube contained 100 μL of cell suspension and 2 μL of antibody. After incubation, 100 μL of PBS was added, and samples were analyzed using a BD FACS Canto II flow cytometer. Data were processed with FlowJo software (BD Biosciences).

### Example 1: Preparation of Primed MSCs

Human mesenchymal stem cells (MSCs), derived from lipoaspirate tissue and commercially obtained from ThermoFisher Scientific (USA) (Catalog number: R7788115), were cultured in AllPhase xeno-free medium (DuoGenic, Taiwan). Cells were seeded at a density of 4,000 cells/cm² and maintained at 37° C. in a humidified atmosphere with 5% CO₂. Upon reaching 80% confluence, MSCs were passaged using Accutase (ThermoFisher) for 3 minutes at 37° C., followed by centrifugation at 1,200 rpm for 5 minutes.

Cells were seeded in 96-well plates at 5,000 cells/well in 100 μL medium and allowed to adhere for 24 hours. The medium was then replaced with fresh medium containing various concentrations of ferroptosis inducers (FINs): erastin (0.1 μM-100 μM), sulfasalazine (0.0156 mM-1 mM) or RSL3 (0.01 μM-10 μM). After 24, 48, or 72 hours of incubation, wells were washed with phosphate-buffered saline (PBS, pH 7.4) and treated with 10 μL of CCK-8 reagent (Dojindo, Japan) per well for 2 hours at 37° C. Absorbance was measured at 450 nm using a microplate reader to determine cell viability and proliferation.

### Example 2: The Effect of Low-Dose FINs on MSC Growth, Cell Viability and Proliferation

To determine whether ferroptosis inducers (FINs) influence mesenchymal stem cell (MSC) proliferation, cells were treated with three different FINs and analyzed for cell viability using the CCK-8 assay.

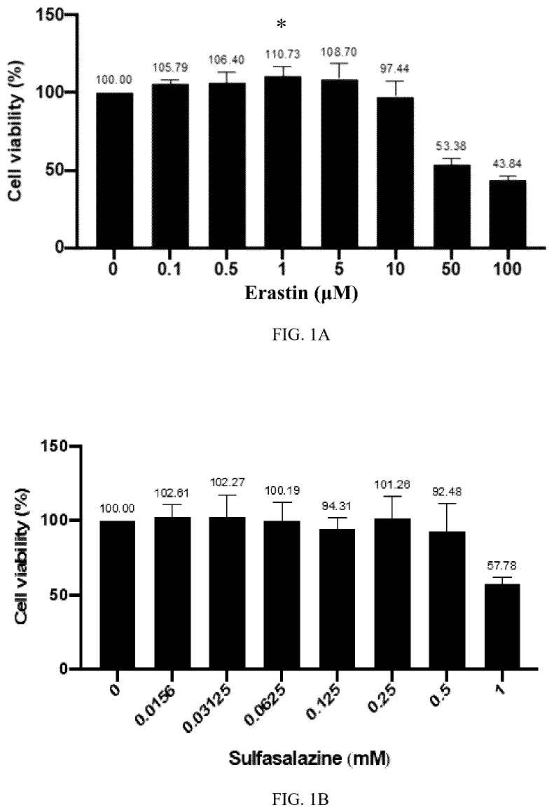

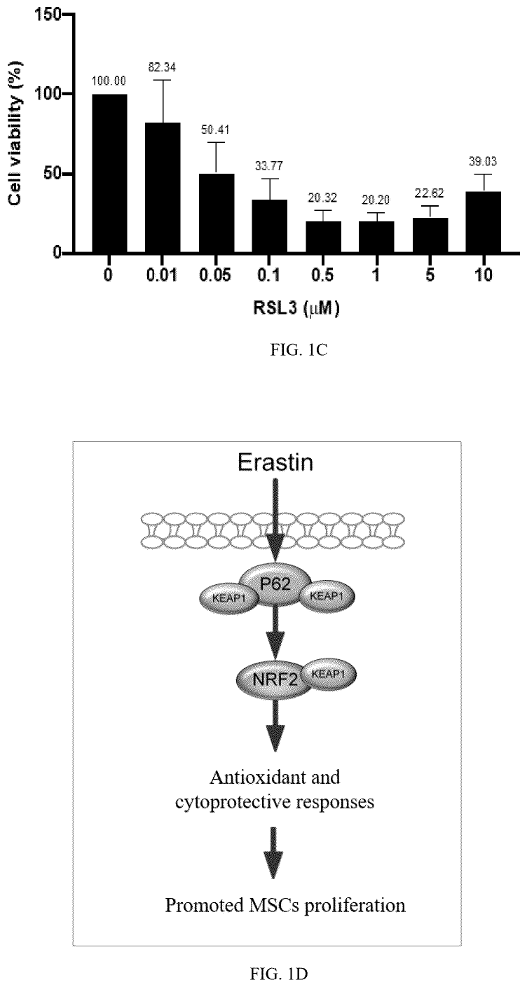

As shown in FIG. 1A and FIG. 1B, among the FINs tested, erastin and sulfasalazine demonstrated a positive effect on MSC proliferation separately. Wherein, MSCs presented an enhancement of cell viability (%) in the condition of 0.1 μM to 5 μM erastin treatment. Wherein, MSCs presented an enhancement of cell viability (%) in the condition of 0.0156 mM to 0.25 mM sulfasalazine treatment. Particularly, erastin significantly enhanced cell viability in a dose-dependent manner, wherein erastin significantly increased MSC growth up to 10.7% (compared to the untreated control, *: p<0.05) at 1 μM concentration after 24 hours of treatment thereof. That is to say, after the FIN treatment (erastin and sulfasalazine respectively), the primed MSCs showed promoted cell viability.

Compared to the low-dose treatment, as shown in FIG. 1A, MSCs presented a significantly decrease of cell viability (%) in the condition of high-dose erastin treatment (10 μM to 100 μM erastin treatment). As shown in FIG. 1B, MSCs presented a significantly decrease of cell viability (%) in the condition of high-dose sulfasalazine treatment (0.5 mM to 1 mM sulfasalazine treatment).

In contrast, as shown in FIG. 1C, RSL3 showed either no effect or cytotoxicity across the concentration range tested.

As above, sub-lethal concentrations of erastin and sulfasalazine can promote MSC expansion respectively, highlighting a functional divergence from its high-dose cytotoxic role. Besides, cell viability results indicated that erastin and sulfasalazine, but not all FINs, promoted MSC growth under conditions of specific low-dose concentration range.

As shown in FIG. 1D, mechanistically, erastin activated the NRF2 pathway via the p62-KEAP1 axis, thereby leading to antioxidant and cytoprotective responses that promoted MSCs proliferation.

### Example 3: Enhancement MSCs Growth Over Time after Low-Dose Erastin Treatment

To further evaluate the proliferative effects of erastin on MSCs, cells were treated with increasing concentrations of erastin (0.5, 1, and 5 μM) and monitored over 72 hours using the CCK-8 assay.

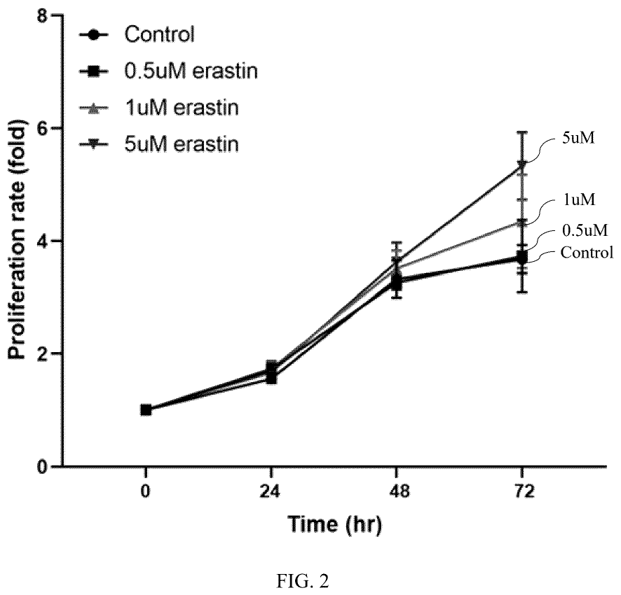

As shown in FIG. 2, results revealed a dose-dependent increase in proliferation. Notably, 5 μM erastin significantly enhanced MSC proliferation rate, achieving a 1.4-fold increase of proliferation rate compared to the untreated control after 72 hours treatment, without inducing cytotoxic effects. Lower concentrations (0.5 and 1 μM) also showed a mild but consistent proliferative advantage. As above, these results indicated that low-dose erastin promoted MSCs growth, particularly under long-term culture conditions.

### Example 4: Erastin Maintains Stemness-Associated Gene Expression and Surface Marker Expression in MSCs

To determine whether low-dose erastin affects the stemness properties of MSCs, it was evaluated the expression of canonical MSC surface markers CD73, CD90, and CD105 at both the transcript level and protein level.

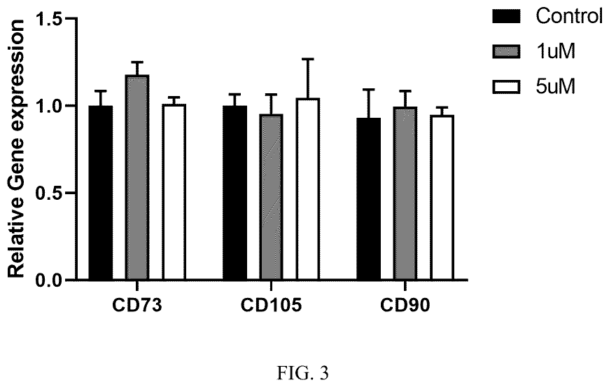

As shown in FIG. 3, quantitative real-time PCR (qRT-PCR) analysis revealed that there were no significant differences in relative gene expression of aforementioned stemness-associated genes, comparing in treatment group (with 1 μM and 5 μM erastin for 72 hours treatment) to untreated controls, thereby indicating that low-dose erastin maintained the stemness properties of MSCs.

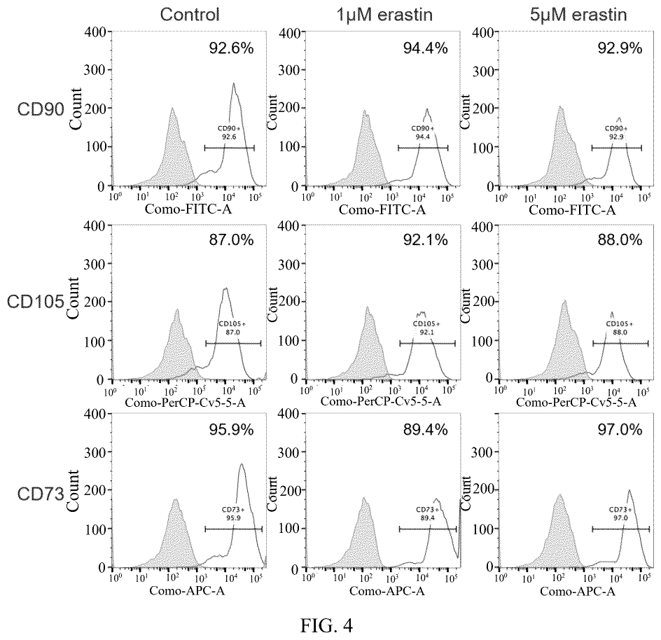

As shown in FIG. 4, high surface expression of CD73, CD90, and CD105 was maintained across all treatment groups (with 1 μM and 5 μM erastin for 72 hours) and untreated control group. Specifically, CD73⁺ expression remained above 89% in all groups and reached 97.0% in the 5 μM erastin treatment group, while CD90⁺ and CD105⁺ cells consistently exceeded 88%. As mentioned above, these results indicated that culture system with exposing to proliferative concentrations of erastin preserved and maintained MSCs stemness and/or identity, thereby indicating use of erastin in culture system aimed at expansion without loss of therapeutic phenotype. The flow cytometry analysis was consistent with the aforementioned qRT-PCR analysis results.

Above all, low-dose FINs could be incorporated into MSC culture systems to promote growth, enhance proliferation, preserve stemness, enhanced oxidative stress tolerance, improved differentiation potential, and elevated immunomodulatory activity. Particularly, treatment with low-dose FINs (such as erastin and/or sulfasalazine) in MSCs culture system, induced sub-lethal oxidative stress without cytotoxicity.

Accordingly, the low-dose FINs offer a novel approach as a priming agent in large-scale stem cell expansion process. And the primed MSCs after low-dose FINs treatment could be applied to transplantation into oxidative and inflammatory microenvironments.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A method of producing an enriched population of isolated and expanded human mesenchymal stem cells (MSCs), the method comprising:
providing an isolated population of human MSCs;
culturing the isolated population of human MSCs in xeno-free medium, and performing subculture upon reaching at least 80% confluence to gain collected population of human MSCs; and
treating the collected population of human MSCs in the presence of priming agent to produce an enriched population of isolated and expanded human MSCs;
wherein the expanded human MSCs are positive for CD73, CD90 and CD105; and
wherein the priming agent includes erastin, sulfasalazine or a combination thereof.

2. The method according to claim 1, wherein the isolated population of human MSCs are adult human MSCs.

3. The method according to claim 1, wherein the isolated population of human MSCs are obtained from bone marrow, fat tissue or peripheral blood.

4. The method according to claim 1, wherein the priming agent is configured to prime the collected population of human MSCs, to produce the primed MSCs.

5. The method according to claim 1, wherein the erastin has a concentration of 0.1 μM to 5 μM.

6. The method according to claim 1, wherein the sulfasalazine has a concentration of 0.0156 mM to 0.25 mM.

7. The method according to claim 1, wherein the treating is carried out for at least 24 hours.

8. The method according to claim 1, wherein a proliferation rate of the expanded MSCs is expanded by at least 1.4-fold after 72 hours of the treating.

9. The method according to claim 1, wherein at least about 89.4% of the expanded MSCs are positive for CD73.

10. The method according to claim 1, wherein at least about 92.9% of the expanded MSCs are positive for CD90.

11. The method according to claim 1, wherein at least about 88.0% of the expanded MSCs are positive for CD105.

12. A method for treating or preventing tissue damage or dysfunction, comprising: administering an effective amount of primed MSCs to a target site of a subject in need thereof, wherein the primed MSCs are produced from steps, the steps comprising:
providing an isolated population of human MSCs;
culturing the isolated population of human MSCs in xeno-free medium, and performing subculture upon reaching at least 80% confluence to gain collected population of human MSCs; and
treating the collected population of human MSCs in the presence of priming agent to produce an enriched population of isolated and expanded human MSCs;
wherein the expanded human MSCs are positive for CD73, CD90 and CD105;
wherein the priming agent includes erastin, sulfasalazine or a combination thereof; and
wherein the target site of the subject is characterized by oxidative stress, inflammation or degenerative condition.

13. The method according to claim 12, wherein the primed MSCs are administrated to the subject by a route of administration selected from the group consisting of transplantation, local injection and systemic infusion.

14. The method according to claim 12, wherein the target site is selected from the group consisting of an osteoblast-associated site, a chondrocyte-associated site and an adipocyte-associated site.

15. The method according to claim 14, wherein the osteoblast-associated site includes: cortical bone, trabecular bone, bone surface, periosteum, bone marrow cavity, osteogenic band or fracture healing site; wherein the chondrocyte-associated site includes: hyaline cartilage, articular cartilage, epiphyseal plate, fibrocartilage, elastic cartilage, cartilage repair site or cartilage of the respiratory tract; and wherein the adipocyte-associated site includes: white adipose tissue, subcutaneous fat, visceral fat, brown adipose tissue, bone marrow fat, fat around organs, mammary gland fat, epicardial fat or perinephric fat.

16. The method according to claim 12, wherein the erastin has an equivalent concentration based on a range of 0.1 μM to 5 μM; and/or wherein the sulfasalazine has an equivalent concentration based on a range of 0.0156 mM to 0.25 mM.

17. A composition comprising primed MSCs and a cell culture medium system, wherein:
the primed MSCs are positive for CD73, CD90 and CD105; and
the cell culture medium system comprises: a xeno-free medium and a priming agent;
wherein the priming agent includes erastin, sulfasalazine or a combination thereof; and
wherein the primed MSCs are obtained from a collected population of human MSCs in the presence of the priming agent to produce primed MSCs.

18. The composition according to claim 17, wherein contacting the collected population of human MSCs with the priming agent enhances a proliferation rate and an oxidative stress tolerance when compared to without the priming agent treatment.

19. The composition according to claim 17, wherein the erastin has a concentration of 0.1 μM to 5 μM; and/or wherein the sulfasalazine has a concentration of 0.0156 mM to 0.25 mM.

20. The composition according to claim 17, wherein at least about 89.4% of the primed MSCs are positive for CD73; wherein at least about 92.9% of the primed MSCs are positive for CD90; and/or wherein at least about 88.0% of the primed MSCs are positive for CD105.
